(19) United States
(12) Patent Application Publication
(10) Pub. No.: US 2009/0131885 A1
(43) Pub. Date: May 21, 2009

(54) Curved Irrigation/Aspiration Needle

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/937,145

(22) Filed: Nov. 8, 2007

Related U.S. Application Data
(60) Provisional application No. 60/864,951, filed on Nov. 8, 2006.
(60) Provisional application No. 60/886,202, filed on Jan. 23, 2007.

(51) Int. Cl. A61M 5/32

(52) U.S. Cl. 604/272

(57) ABSTRACT

An irrigation/aspiration needle has needle segments offset with one another and with the needle amount to position the needle port more accurately and conveniently. The port is preferably positioned at various selected sites on the needle.

### PRIORITY

This application claims priority from U.S. Patent Application Ser. No. 60/864,951, filed Nov. 8, 2006 and U.S. Patent Application Ser. No. 60/886,202, filed Jan. 23, 2007, both of which are incorporated herein by reference.

### FIELD OF THE INVENTION

The present invention relates to instruments used in eye surgery and, more particularly, to an irrigation/aspiration needle used to remove particles from the capsular bag after removal of a lens.

### BACKGROUND OF THE INVENTION

The lens of a human eye is held within a capsular bag positioned behind the iris in the anterior chamber of the eye. When the lens becomes damaged or diseased a common surgical technique is to remove the lens and replace it with an artificial intraocular lens (IOL). Removal of the lens is commonly carried out by phacoemulsification, that is, using a needle to which vibrational electrical energy is transmitted by a phacoemulsification hand piece.

During phacoemulsification, the lens is broken into fragments and the fragments are emulsified and then removed from the capsular bag by aspirating the fragments through a canula formed as part of the phacoemulsification needle. After the lens has been removed, the capsular bag must be cleaned in order to prepare it for the insertion of an IOL. In particular, epithelial and cortical tissue fragments must be removed from the floor and perimeter of the capsular bag.

Complete removal of the cortex is important for several reasons. If the cortex is not completely removed it may cause post-operative inflammation and an increase in intraocular pressure. Incomplete removal of the cortex may also cause decentration or tilting of the IOL which, in turn, would cause a postoperative refractive error or induced astigmatism. This is especially important if the IOL is a multi-focal type. Incomplete removal of the cortex may also result in the formation of another cataract which would impair vision.

It is difficult to obtain complete cortical removal and aspiration using a conventional coaxial irrigation/aspiration tip having an irrigation sleeve on it. For example, if the surgeon places the irrigation/aspiration tip close to the sub-incisional cortex, irrigation fluid passing through the sleeve will end up outside the anterior chamber and cause collapse of the eyeball. Alternatively, if the surgeon tries to remove the sub-incisional portion of the cortex by placing the irrigation/aspiration tip vertically, great stress is placed on the incision which may cause insufficient sealing at the end of the surgery. This may result in endophthalmitis.

The present invention has an ergonomically curved tip designed to reach any part of the capsular bag to remove the cortex without stressing the incision.

Because the tip is gently curved, it can easily reach the sub-incisional cortex through a micro-coaxial incision. This type of curved design is suited for removing E-type lens epithelial cells found on the formax of the capsular bag and may later cause a cataract. The tip diameter of the present invention may be as small as 0.7 mm which makes it possible to manipulate through a sub-2 mm micro coaxial incision using a nano or ultra irrigation sleeve. Adequate irrigation can be obtained in the large space between the tip and the sleeve to maintain a stable anterior chamber pressure during surgery.

The aspiration port of the present invention is preferably oval which enhances the particle aspiration as compared to a more conventional round port. The tip surface may also be sand blasted and can then be used to polish the anterior capsule to remove fine cortical residue as well as any remaining viscoelastic material present after implanting the lens. To aspirate the viscoelastic material, the tip is introduced beneath the IOL accomplishing aspiration without stressing the capsular bag or the ciliary zonules.

Heretofore it has been common to use an I/A needle which is straight along its entire length in order to effect removal of the tissue fragments. This requires movement of the needle across the floor and around the periphery of the capsular bag, a range of motion to which the straight needle is not particularly well-suited. The prior art demonstrates examples of curved or curvable needles which attempt top improve maneuverability within the eye during surgery.

European Patent Application EP 1,707,166 (Ghamnoun) teaches and describes an irrigation tip used with a surgical hand piece. The tip has a portion of which is curved at a single bend.

U.S. Pat. No. 7,037,296 (Kadziauskas) teaches and describes a curved multi-purpose phacoemulsification needle having a curved distal tip portion. The curve in Kadziauskas appears to be a single curve.

U.S. Pat. No. 5,217,465 (Steppe) teaches and describes a flexible and steerable aspiration tip for microsurgery formed as an aspiration tip used with a phacoemulsification hand piece. The tip has a hollow flexible tubular member within which a coil spring is disposed. Pulling the spring allows the tip to be bent into a curve and held there. The tip in Steppe, when pulled into an arc, forms a single curvature bend.

U.S. Published Patent Application US 2006/0189948 (Boukhny) teaches and describes a phacoemulsification tip having a single fixed bend.

In U.S. Pat. No. 5,217,465, the patentee describes the difficulty in removing the cortex from the capsular bag:

“In an effort to remove the cortex from the location directly below the incision, several complicated and difficult maneuvers using the straight and rigid irrigation/aspiration tip must be employed. In one maneuver, the iris is drawn out of the wound using smooth forceps while the irrigation/aspiration tip is inserted and exposed cortex is engaged by aspiration. These maneuvers are difficult to execute since visualization of the underlying cortex through the iris is impossible and, in certain cases with a constricted iris, even more difficult to perform. As a result of these different maneuvers, excess trauma may result in the surrounding ocular tissue including exposing and/or weakening of the zonules 120 or enlargement of the limbal incision with subsequent possible collapse of the anterior chamber 109.”

I have determined that providing an irrigation/aspiration tip with multiple curved portions allows the surgeon to maneuver the tip to contact a floor of the capsular bag about its periphery. In particular, I have found that providing a first, relatively small radius bend in the tip proximate the distal end of the tip and a second, larger radius bend intermediate the first bend and the proximal end of the tip allows the tip to be maneuvered to follow the floor and periphery of the capsular bag and to efficiently remove cortical tissue. Placement of the irrigation/aspiration port at various locations on the surface of the needle also enhances the maneuverability of the instrument.

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further aspects of the present invention will best be understood by consideration of the accompanying drawings in which:

FIG. 1 is a view of a prior art irrigation/aspiration tip;

FIG. 2 is an illustration of a prior art phacoemulsification needle;

FIG. 3 is an illustration of a prior art steerable needle tip;

FIG. 4 is an illustration of a prior art curved phacoemulsification needle;

FIG. 5 is a perspective view of an example of the present invention;

FIG. 6 is a lateral schematic view of FIG. 5;

FIG. 7 is a view of FIG. 6 displaying dimensional data;

FIG. 8 is a perspective view of an example of the present invention;

FIG. 9 is a perspective view an example of the present invention;

FIG. 10 is a lateral view of FIG. 9:

FIG. 11 is a second perspective view of FIG. 9;

FIG. 12 is a an end view of FIG. 11;

FIG. 13 is a partial sectional view of the tip of FIG. 12; and

FIG. 14 is a lateral schematic view with dimensional information.

### DETAILED DESCRIPTION OF THE DRAWINGS

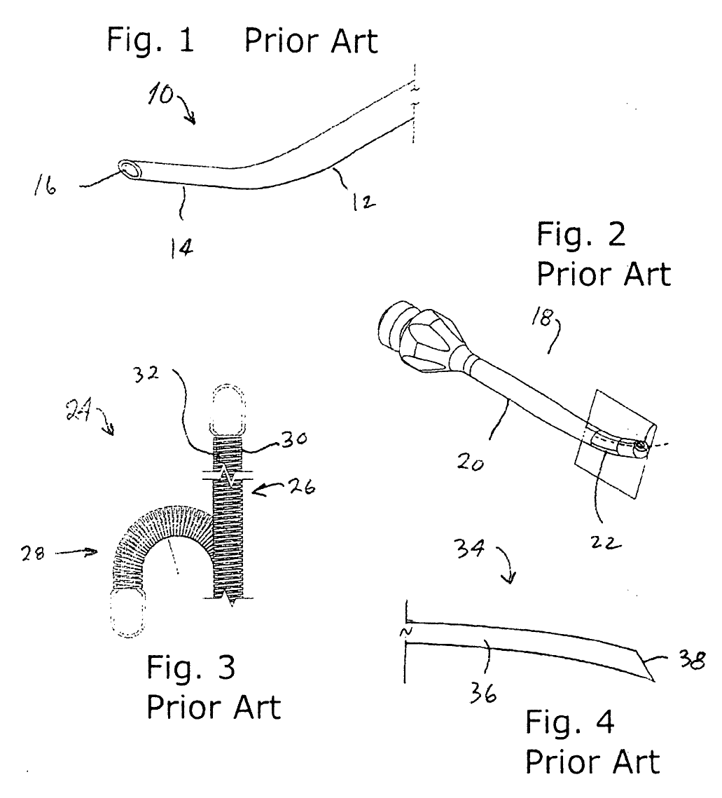

Referring now to FIG. 1, the numeral 10 identifies a prior art irrigation tip as described in European Patent Application EP 1,707,166. Tip 10 has a proximal shaft 12 and a distal shaft 14 with the distal shaft 14 terminating in a port 16. As seen in FIG. 1, distal shaft 14 is bent with respect to proximal shaft 12 to form a single curve.

Referring now to FIG. 2, numeral 18 identifies a phacoemulsification needle as described in U.S. Pat. No. 7,037,296. Needle 18 has a needle body 20 and a tip portion 22 formed at an angle to needle body 20 with a single curve.

Referring now to FIG. 3, numeral 24 identifies a steerable and adjustable aspiration tip as described in U.S. Pat. No. 5,217,465. Tip 24 is shown in a first, straight position 26 and in a second curved position 28. Tip 24 has a flexible outer tube 30 within which a coil spring 32 is disposed and further includes means for causing tip 24 to curve from the position 26 to position 28 (and other intermediate positions) during surgery. As shown in FIG. 3, a single radius of curvature is obtained.

Referring now to FIG. 4, the numeral 24 identifies a prior art phacoemulsification tip as shown and described in U.S. Patent Application Publication 2006/0189948. Tip 34 is depicted as having a single curved and rigid segment 36 which is hollow and through which tissue may be aspirated through mouth 38.

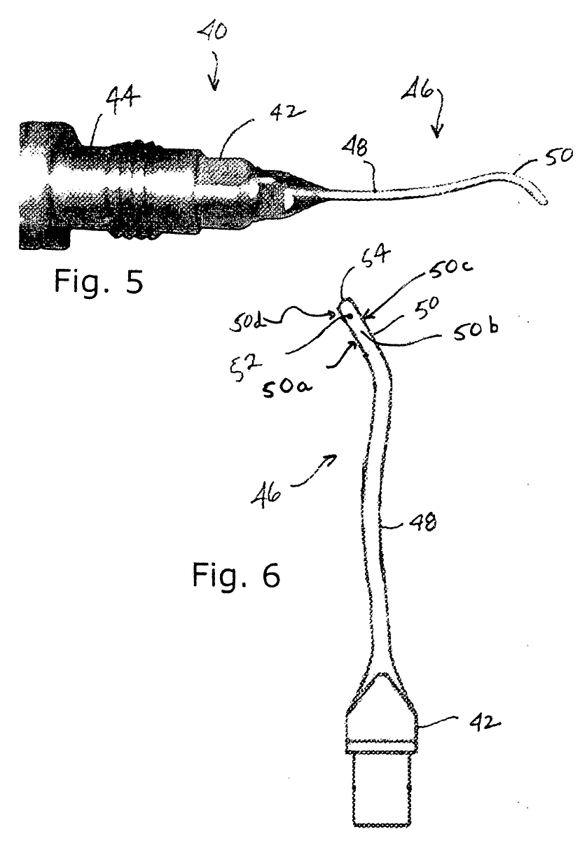

Referring now to FIG. 5, the numeral 40 identifies an irrigation/aspiration tip embodying features of the present invention. Tip 40 has a tip mount 42 allowing it to be attached to hand piece 44. Extending from mount 42 is a tip shaft 46. In the present example, tip 46 has a first, proximal shaft segment 48 which is curved in a first direction and a second, distal shaft segment 50 which is curved in a second direction. In the example shown, shaft segments 48 and 50 are curved in opposite directions with respect to each other.

Referring now to FIG. 6, a lateral view of the tip of FIG. 5 is shown. In this example, shaft 46 is planar, meaning that an axis drawn along tip 46 from shaft segment 50 and along shaft segment 48 to mount 42 would lie in a single plane. It is to be understood that tip 46 is hollow and communicates with a passageway within mount 42 and to another passageway within hand piece 44.

To further describe shaft segment 50 as seen in FIG. 6, the outer surface of shaft segment 50 has a first lateral portion 50a extending generally along that portion of shaft segment 50 that is curved inward or toward shaft segment 48 at an angle less than 180°, a third lateral portion 50c is generally opposite surface 50a, and second and fourth lateral portions 50b, 50d which are opposite one another and extend between lateral portions 50a and 50c.

Referring again to FIG. 6, a port 52 is shown positioned proximate closed end 54 on lateral surface portion 50b of shaft segment 50. In this example, port 52 would be in a plane parallel to the plane described above which would contain the axis of tip 46.

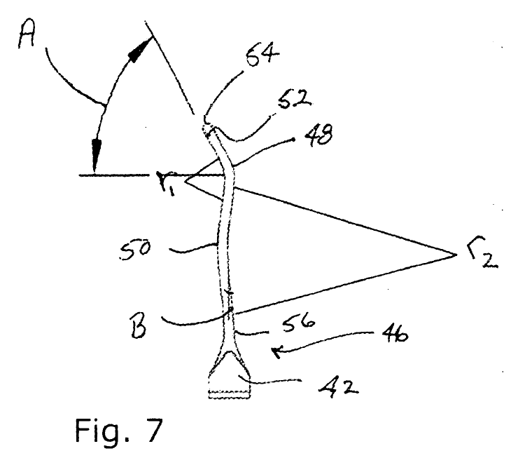

Referring now to FIG. 7, an example of tip 46 is shown wherein the radius of curvature of shaft segment 48 is r₁ and the radius of curvature of shaft segment 50 is r₂. In the example shown, r₂ is greater than r₁

Also, as seen in FIG. 7, shaft segment 50 is offset from shaft segment 48 by an angle A of about 55°.

In the example in FIG. 7, tip 46 includes a mount shaft segment 56 extending from mount 42 and forming an offset angle B of about 10° with shaft segment 50.

In one example of the present invention r₁is about 6 mm and r₂ is about 22 mm. The selected radii of curvature and the placement of port 52 may be altered or modified if desired.

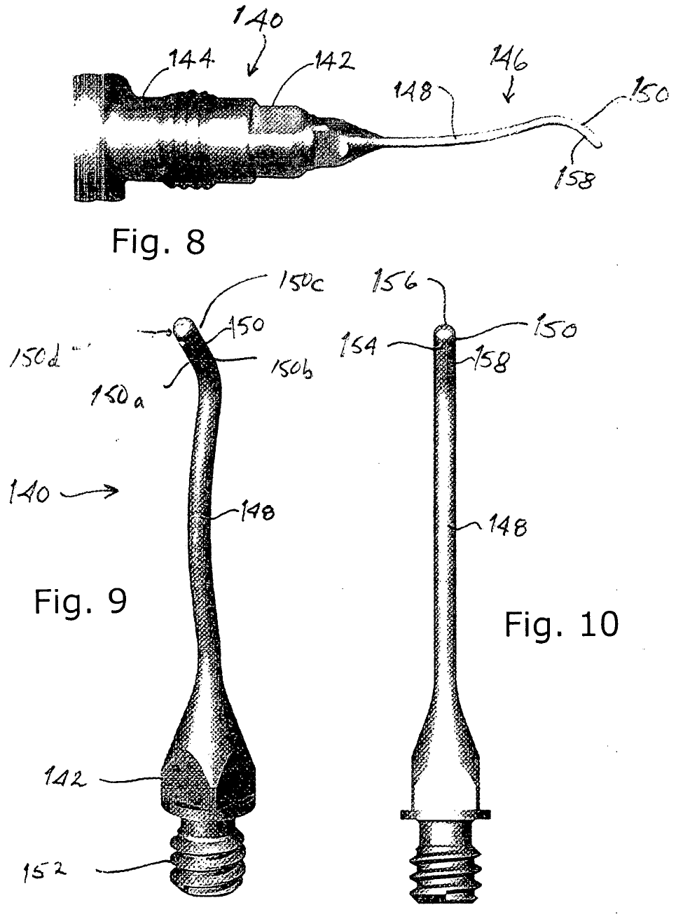

Referring now to FIG. 8, the numeral 140 identifies a hollow irrigation/aspiration tip embodying features of another example of the present invention. Tip 140 has a tip mount 142 allowing it to be attached to a hand piece 144. Extending from mount 142 is a tip shaft 146. In the present example, tip shaft 146 has a first, proximal segment 148 which is curved in a first direction and a second, distal shaft segment 150 which is curved in a second direction. Other curved shaft segments are identified herein as well. In the example shown, shaft segments 148 and 150 are curved in opposite directions with respect to each other.

Referring now to FIG. 9, a perspective view of tip 140 is shown as removed from handpiece 144. Extending from mount 142 is a threaded segment 152, used to thread tip 140 onto handpiece 144.

To further describe shaft segment 150 as seen in FIG. 9, the outer surface of shaft segment 150 has a first lateral portion 150a extending generally along that portion of shaft segment 150 that is curved inward or toward shaft segment 148 at an angle less than 180°, a third lateral portion 150c that is generally opposite surface 150a, and second and fourth lateral portions 150b, 150d which are opposite one another and extend between lateral portions 150a and 150c.

Referring now to FIG. 10 it can be seen that in the example shown in FIG. 9 both shaft segment 148 and shaft segment 150 are coplanar such that tip 140 appears to be straight. A port 154 is shown positioned proximate closed end 156 of shaft segment 150. In this example, port 154 lies on the “inward” wall 158 of shaft segment 150, that is, that part of segment 150 that is curved toward and closest to shaft segment 148. It is to be understood that port 154 communicates with a passageway within hollow tip 140 which, in turn, communicates to a passageway through mount 142 and to another passageway within handpiece 144.

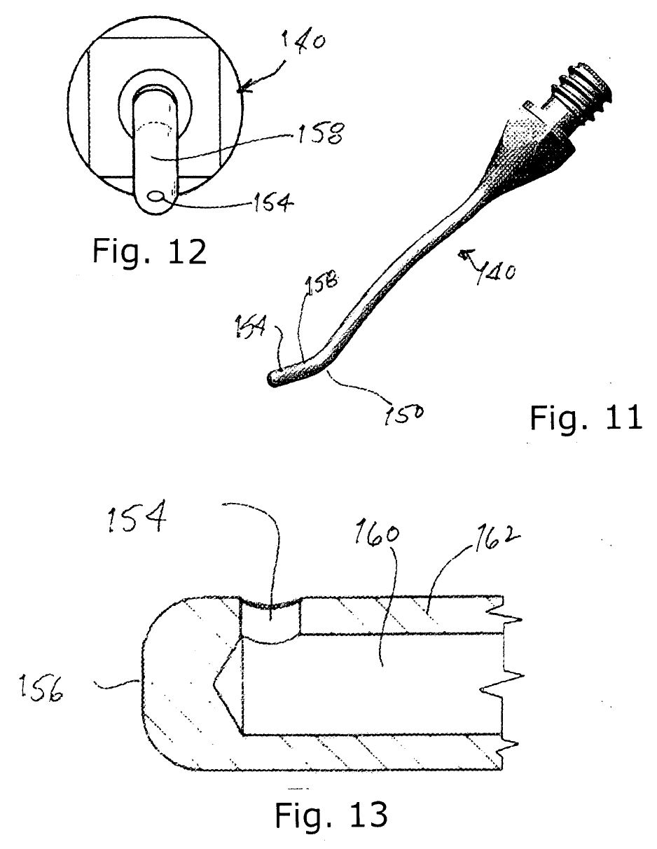

Referring to FIG. 11, tip 140 is shown in a different perspective view, offering a different view of the placement of port 154 on shaft segment 150.

Referring now to FIG. 12, an end view of tip 140 is shown with port 154 seen on inward wall 158.

Referring now to FIG. 13, the end of shaft segment 150 is shown in partial section. Hollow tip 140 has an interior passage 160 which, in this view, is defined by cylindrical tip wall 162 and tip end 156. Port 154 is formed through wall 162 and communicates with passage 60, allowing fluid to pass through passage 160 and port 154 into the eye.

At least one irrigation/aspiration port must be formed on tip 140. Port 154 has been illustrated herein as positioned along inward wall 158, but can be formed anywhere along shaft segment 150 as desired. For example, a port may be formed directly through end 156 into passage 160, and may also be formed at other selected sites, either on inward wall 158 or along another selected wall segment. Multiple ports can also be formed as needed or desired. Forming port 154 along inward wall 158 makes tip 140 particularly convenient to reach the sub-incisional portion of the capsular bag.

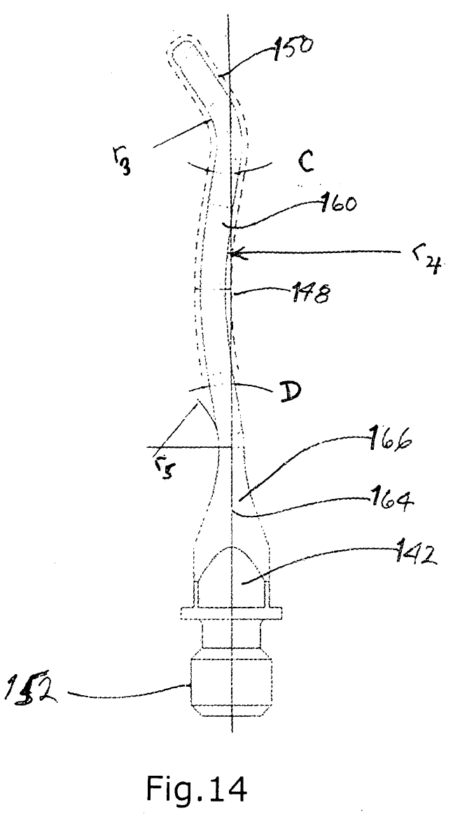

Referring now to FIG. 14, an example of tip 140 is shown wherein the radii of curvature of shaft segment 148, shaft segment 150 and various other radii defining the shape of tip 140 are identified.

In FIG. 14, radius r₃ is the radius of curvature of shaft segment 150 at shaft segment 148, while the radius of curvature of shaft segment 148 is r₄. In the example shown, r₄ is greater than r₃.

Also, as seen in FIG. 14, shaft segment 148 is offset from mount 142 with a radius r₅ sufficient to create an offset of about 10° with tip axis 164, while shaft segment 150 is offset from shaft segment 148 at radius r₃ by an angle C of about 55°. Shaft segment 148 is also offset at radius r₅ to create an offset angle D of about 10° with axis 164.

In one example of the present invention r₃ is about 6 mm and r₄ is about 22 mm. The selected radii of curvature and the placement of port 152 may be altered or modified if desired.

FIG. 14 also illustrates passage 160 communicating with throat 166, extending through mount 142 and threaded segment 152 to communicate with a handpiece.

I claim:

1. An aspiration/irrigation needle, said needle comprising:
a hollow needle shaft having a shaft wall,
said hollow needle shaft having a distal end and a proximal end,
said shaft wall defining a fluid passageway;
means for attaching said needle shaft to a fluid flow line;
said needle shaft having a first shaft segment beginning at said proximal end,
said proximal end fluid-tightly attached to said attachment means;
said needle shaft having at least a second shaft segment contiguous with and angled from said first shaft segment at a first offset,
said second shaft segment having a closed end terminating at said distal end; and
at least one port formed on said second shaft segment and communicating with said fluid passageway.

2. The apparatus as recited in claim 1 wherein said first offset is a curve formed in said needle shaft.

3. The apparatus as recited in claim 2 wherein said first offset has a radius of about 6 mm.

4. The apparatus as recited in claim 2 wherein said first and second shaft segments form an angle of about 55° with respect to one another.

5. The apparatus as recited in claim 1 wherein said first shaft segment is curved.

6. The apparatus as recited in claim 5 wherein said first shaft segment has a radius of about 22 mm.

7. The apparatus as recited in claim 1 wherein said at least second shaft segment comprises:
a first outer wall surface,
said first outer wall surface being that part of said at least second shaft segment that is angled toward said first shaft segment at an angle of less than 180°;
a third outer wall surface generally opposite to said first outer wall surface;
a second outer wall surface extending between said first and third outer wall surfaces;
a fourth outer wall surface extending between said first and third outer wall surfaces; and
said at least one port is formed through one of said outer wall surfaces.

8. The apparatus as recited in claim 1 wherein said at least one port is formed through said second shaft closed end.

9. The apparatus as recited in claim 1 wherein said port is oval.

10. An aspiration/irrigation needle, said needle comprising:
a hollow needle shaft having a shaft wall,
said hollow needle shaft having a distal end and a proximal end,
said shaft wall defining a fluid passageway;
means for attaching said needle shaft to a fluid flow line;
said needle shaft having a first shaft segment beginning at said proximal end,
said proximal end fluid-tightly attached to said attachment means,
said first shaft segment angled from said attaching means at a first offset;
said needle shaft having at least a second shaft segment contiguous with and angled from said first shaft segment at a second offset,
said second shaft segment having a closed end terminating at said distal end; and
at least one port formed on said second shaft segment and communicating with said fluid passageway.

11. The apparatus as recited in claim 10 wherein said first second is a curve formed in said needle shaft.

12. The apparatus as recited in claim 11 wherein said second offset has a radius of about 6 mm.

13. The apparatus as recited in claim 11 wherein said first and second shaft segments form an angle of about 55° with respect to one another.

14. The apparatus as recited in claim 10 wherein said first shaft segment is curved.

15. The apparatus as recited in claim 14 wherein said first shaft segment has a radius of about 22 mm.

16. The apparatus as recited in claim 10 wherein said at least second shaft segment comprises:
a first outer wall surface,
said first outer wall surface being that part of said at least second shaft segment that is angled toward said first shaft segment at an angle of less than 180°;
a third outer wall surface generally opposite to said first outer wall surface;
a second outer wall surface extending between said first and third outer wall surfaces;
a fourth outer wall surface extending between said first and third outer wall surfaces; and
said at least one port is formed through one of said outer wall surfaces.

17. The apparatus as recited in claim 10 wherein said at least one port is formed through said second shaft closed end.

18. The apparatus as recited in claim 10 wherein said first offset is about 10°.

19. The apparatus as recited in claim 10 wherein said port is oval.
